(19) United States
(12) Patent Application Publication
(10) Pub. No.: US 2022/0000774 A1
(43) Pub. Date: Jan. 6, 2022

(54) SUPPOSITORY FORMULATIONS HAVING CANNABINOID

(71) Applicant: Columbia Care LLC, New York, NY (US)

(72) Inventor: Aaron Michael Dely, Lighthouse Point, FL (US)

(21) Appl. No.: 17/291,405

(22) PCT Filed: Nov. 7, 2019
(86) PCT No.: PCT/US2019/060307

§ 371 Date: May 5, 2021

Related U.S. Application Data
(60) Provisional application No. 62/757,026, filed on Nov. 7, 2018.

(51) Int. Cl. A61K 9/02; A61K 36/185; A61K 31/352; A61K 31/05; A61K 47/10; A61K 47/38

(52) U.S. Cl. CPC A61K 9/02; A61K 36/185; A61K 47/38; A61K 31/05; A61K 47/10; A61K 31/352

(57) ABSTRACT

The invention relates to cannabinoid suppository formulations and methods associated therewith. Specifically, the invention relates to a rectal or a vaginal suppository formulation having a cannabinoid in combination with a moldable polymer.

### CROSS-REFERENCE TO RELATED APPLICATIONS

This application claims the benefit of U.S. Provisional Application No. 62/757,026, filed Nov. 7, 2018, the entire contents of which are incorporated by reference herein.

### FIELD OF THE INVENTION

The invention relates to cannabinoid suppository formulations and methods associated therewith. Specifically, the invention relates to a rectal or a vaginal suppository formulation having a cannabinoid in combination with a moldable polymer.

### BACKGROUND OF THE INVENTION

The medicinal and psychoactive properties of the cannabis plant have been known for centuries. While it has been illegal in many countries, there is a growing populous to lobby for legalization of its use, especially for medicinal purposes.

Cannabis is believed to provide benefits in the treatment of multiple disorders with safer and fewer serious side effects than most prescription drugs currently used as antiemetics, muscle relaxants, hypnotics and analgesics. A disadvantage in treating patients with cannabis is the psychoactive effect, especially in “naive” cannabis users. Furthermore, there have been reports of unpleasant reactions to cannabis, such as anxiety, panic or hallucinations. It is believed that the undesirable side effects are most commonly associated with higher doses of cannabis, and are related to the difficulty in controlling the dosage when the drug is smoked or eaten in cannabis-enriched confectionaries.

Cannabis has also been used to treat the symptoms in patients suffering from serious medical conditions. For example, cannabis has been used to alleviate symptoms associated with cancer, anorexia, AIDS, chronic pain, muscle spasticity, glaucoma, arthritis, migraine and many other illnesses. Cannabis is recognized as having anti-emetic properties and has been successfully used to treat nausea and vomiting in cancer patients undergoing chemotherapy. Cannabis has also been reported in treating the weight loss syndrome of AIDS and for the treatment of glaucoma by reducing intraocular pressure. Cannabis is also known for its muscle relaxing and anti-convulsant effects.

The most prevalent mode of administration of medical cannabis is by smoking. This mode of administration can have adverse effects on the lungs. Cannabis smoke carries more tar and other particulate matter than tobacco, and may be a cause of lung diseases including lung cancer. Furthermore, many patients find the act of smoking unappealing, as well as generally unhealthy.

Accordingly, there is significant interest in developing other means to administer cannabis to patients.

To date, no suppository formulation exists for cannabis. There remains an unmet need for suppository formulations for administering cannabis or cannabinoid.

### SUMMARY OF THE INVENTION

In one aspect, the invention provides a suppository formulation comprising: at least one cannabinoid or a derivative thereof and at least one moldable polymer. In certain embodiments, the suppository formulation is a rectal or a vaginal suppository formulation.

In certain embodiments, the formulation includes a first cannabinoid (e.g., THC or THCa) and a second cannabinoid (CBD or CBDa). In other embodiments, the formulation includes a first polymer (e.g., polyethylene glycol 1450) and a second polymer (e.g., polyethylene glycol 4000).

In another aspect, the invention provides a method for treating a disease or disorder in a subject, the method comprising administering, for example, rectally or vaginally administering, a suppository formulation of the invention.

In yet another aspect, the invention provides a method for manufacturing a suppository formulation of the invention.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, other embodiments include from the one particular and/or to the other particular value. Similarly, when values are expressed as approximations, by use of the antecedent “about,” it is understood that the particular value forms other embodiments. All ranges are inclusive and combinable.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder. In some embodiments, this condition, disease or disorder can be sleep apnea.

The terms “subject,” “individual,” and “patient” are used interchangeably herein, and refer to an animal, for example a human, to whom treatment, including prophylactic treatment, with the pharmaceutical formulation according to the present invention, is provided.

In one aspect, provided herein is a suppository formulation comprising at least one cannabinoid or a derivative thereof and at least one moldable polymer. The formulation can be an insertable or implantable formulation. In certain exemplary embodiments, the suppository formulation is a rectal suppository formulation. In other exemplary embodiments, the suppository formulation is a vaginal suppository formulation.

Some embodiments of the invention relate to suppository compositions of one or more cannabinoids to provide a release, for example, the sustained release of said one or more cannabinoids. The cannabinoids are easily prepared and formulated to provide consistent therapeutically effective dosage forms from lot to lot. In some embodiments, a “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated. The skilled worker can readily determine the effective amount for a given situation by routine experimentation.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

The inventor of the instant application surprisingly and unexpectedly discovered a suppository formulation which includes, in part, one or more cannabinoids in combination with one or more polymers.

In some embodiments, the cannabinoid is an extract from a cannabis plant (e.g., a cannabinoid extract). In some embodiments, the cannabinoid is a cannabinoid extract that contains a combination of at least two of the following: Tetrahydrocannabinolic acid (THCa), Cannabidiolic acid (CBDa), Cannabinolic acid (CBNa), Cannabichromenic acid (CBCa), Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CB T), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM) and derivatives thereof. The cannabinoid may be natural or synthetic.

The methods of making cannabinoids extract is well known in the art. The cannabis plants are grown, harvested, and the cannabinoids are extracted through, for example, a CO₂ extraction process.

In some embodiments, the formulation has a combination of at least two cannabinoids. In some embodiments, the formulation comprises a combination of at least two cannabinoids. In some embodiments, the suppository formulation includes a first cannabinoid and a second cannabinoid. In some embodiments, the two cannabinoids are selected from THCa, CBDa, CBNa, CBCa, CBD, CBG, CBC, CBN, CBE, iso-THC, CBL, CBT, CBV, THCV, CBDV, CBCV, CBGV, CBGM, and derivatives thereof. In some embodiments, the first and second cannabinoids are in a ratio ranging from about 1:1 to about 20:1. In some embodiments, the first and second cannabinoids are present in a 1:1 proportion by weight. In some embodiments, the at least two cannabinoids are present in a 1:1 proportion by weight. In other embodiments, the first and second cannabinoids are present in a 10:1 proportion by weight. In other embodiments, the at least two cannabinoids are present in a 10:1 proportion by weight. In yet other embodiments, the first and second cannabinoids are in a 20:1 proportion by weight. In yet other embodiments, the at least two cannabinoids are present in a 20:1 proportion by weight.

In certain preferred embodiments, the first cannabinoid is THC or THCa and the second cannabinoid is CBD or CBDa (e.g., the first cannabinoid is THC and the second cannabinoid is CBD, or the first cannabinoid is THCa and the second cannabinoid is CBDa). In certain preferred embodiments, the two cannabinoids are THC and CBD. In certain preferred embodiments, the two cannabinoids are THCa and CBDa.

In certain exemplary embodiments, the first cannabinoid can be present in an amount ranging from about 0.1 mg to about 200 mg. For example, the first cannabinoid can be THC or THCa present in an amount ranging from about 0.1 mg to about 200 mg. The second cannabinoid also can be present in an amount ranging from about 0.01 mg to about 200 mg. For example, the second cannabinoid can be CBD or CBDa present in an amount ranging from about 0.01 mg to about 200 mg.

In another non-limiting example, at least one cannabinoid can be THC or THCa present in an amount ranging from about 0.1 mg to about 200 mg.

In another non-limiting example, at least one cannabinoid can be CBD or CBDa present in an amount ranging from about 0.01 mg to about 200 mg.

In some embodiments, the cannabinoids are in equal proportion such as, for example, 2.5 mg THC to 2.5 mg CBD. Other embodiments include proportions of THC/CBD of 0.25 mg THC to 5.0 mg CBD or 5.0 mg THC to 0.25 mg CBD.

In some embodiments, cannabinoid of the invention is any member of a group of substances that are structurally related to tetrahydrocannabinol and that bind to a cannabinoid receptor such as CB1 or CB2 or both (“THC”). The cannabinoid can be a naturally occurring compound (e.g. present in Cannabis), a compound metabolized by a plant or animal, or a synthetic derivative.

The cannabinoid may be included in its free form, or in the form of a salt; an acid addition salt of an ester; an amide; an enantiomer; an isomer; a tautomer; a prodrug; a derivative of an active agent of the invention; different isomeric forms (for example, enantiomers and diastereoisomers), both in pure form and in admixture, including racemic mixtures; or enol forms.

The cannabinoids of the invention are further meant to encompass natural cannabinoids, natural cannabinoids that have been purified or modified, and synthetically derived cannabinoids. Methods for purifying cannabinoids, obtained from plant material, are well known in the art and fully described in, for example, United States Patent Application Publication 2005/0266108, which is incorporated by reference herein in its entirety.

The cannabinoids of the invention can be any of 9-tetrahydrocannabinol, 8-tetrahydrocannabinol, (+)-1,1-dimethylheptyl analog of 7-hydroxy-delta-6-tetrahydrocannabinol, 3-(5′-cyano-1′,1′-dimethylpentyl)-1-(4-N-morpholinobutyryloxy) delta 8-tetrahydrocannabinol hydrochloride], dexanabinol, nabilone, levonantradol, and N-(2-hydroxyethyl) hexadecanoamide. In some embodiments, the cannabinoids of the invention can be any of the non-psychotropic cannabinoid 3-dimethylnepty 11 carboxylic acid homologine 8, and delta-8-tetrahydrocannabinol.

In some embodiments, the cannabinoid extract comprises cannabinoids tincture. The term “tincture”, as used herein, refers to an alcoholic extract of plant material. Methods of tincture preparation are well known in the art and are described, for example, in the U.S. patent application U.S. Pat. No. 9,468,865, hereby incorporated by reference in its entirety. In certain embodiments, the method of tincture preparation according to the present invention comprises the steps of combining medium chain triglycerides and polysorbate in a mixing vessel to form a first combination; mixing the first combination; adding the cannabinoid extract to the first combination to form a second combination; mixing the second combination; adding 200 proof ethanol to the second combination to form a third combination; and mixing the third combination for an extended period of time in order to obtain the cannabinoids tincture. The duration of the final mixing step may vary depending on many factors, and can be easily optimized by a skilled artisan. In some embodiments, the final mixing step ranges from about 5 to about 60 minutes. In some embodiments, the final mixing step ranges from about 10 to about 50 minutes. In some embodiments, the final mixing step ranges from about 15 to about 45 minutes. In some embodiments, the final mixing step ranges from about 20 to about 40 minutes.

In another aspect, the suppository formulation of the invention includes one or more moldable polymers. In certain embodiments, the polymer (e.g., the one or more moldable polymers) comprises polyethylene glycol (PEG). In certain such embodiments, the polymer (e.g., the one or more moldable polymers) is PEG. The molecular weight of PEG may range from about 100 to about 10,000, preferably from about 1000 to about 5000. In certain embodiments, the PEG is characterized by a molecular weight that ranges from about 1000 to 5000. In certain embodiments, PEG is PEG 1450. In other embodiments, PEG is PEG 4000.

In some embodiments, the suppository formulation comprises a combination of at least two polymers (e.g., at least two moldable polymers). In some embodiments, the suppository formulation has a combination of at least two polymers (e.g., at least two moldable polymers). In some such embodiments, the first polymer is PEG 1450 and the second polymer is PEG 4000.

The first and second polymers can be present in a formulation at a ratio ranging from about 2:1 to about 5:1. In certain embodiments, the first and second polymers are present in the formulation at a ratio 3:1.

In certain embodiments, the suppository formulation of the invention further comprises a terpene. Examples of a terpene include, for example, but not limited to, beta-myrcene, limonene, beta caryopyllene, caryopyllene oxide, terpineol, citronellol, linalool, humulene, beta-amyrin, and cycloartenol.

The suppository formulation of the invention may also include additional ingredients such as solvents, carriers or excipients.

In some embodiments, the formulation further comprises a solvent. In some such embodiments, the solvent comprises ethanol, methanol, isopropanol, chloroform, propylene glycol, polyethylene glycol, glycerine, limonene, myrcene, linalool, alpha bisabolol, delta 3 carene, borneol, alpha-pinene, beta-pinene, eucalyptol, terpineol, caryophyllene, camphene, or combinations thereof. In some such embodiments, the solvent is ethanol.

In some embodiments, the formulation further comprises a pharmaceutically acceptable carrier. In some such embodiments, the carrier comprises cellulose, microcrystalline cellulose, silicified microcrystalline cellulose, starch, pregelatinized starch, dicalcium phosphate, tricalcium phosphate, or mixtures thereof. In certain embodiments, the carrier is microcrystalline cellulose. In other embodiments, the carrier may be comprised of a water-soluble sugar or sugar alcohol. Examples include, but not limited to, lactose, sucrose, dextrose, polydextrose, fructose, maltose, maltodextrin, dextrate, dextrin, lactitol, mannitol, erythritol, maltitol, sorbitol, or xylitol, and mixtures thereof.

In some embodiments, the formulation of the invention further comprises a film coating. Materials for film coating include, but not limited to, for example, coating base and coating ingredients. These materials are used in an amount which is commonly used in the pharmaceutics formulation area. Numerous types of film coating are described in the art and, for example in the U.S. Pat. No. 8,541,024, hereby incorporated by reference in its entirety. An example of a film forming polymer used to form the film coating includes, for example, a cellulose polymer. The cellulose polymer preferably has a molecular weight of 7,500 to 120,000 and a viscosity of 1.5 to 20 mm²/s (25 C.°). For example, cellulose ether polymer which is one of cellulose polymer includes, for example, hydroxylpropyl methylcellulose (HPMC), hydroxylpropyl cellulose, methylcellulose, or ethylcellulose, among which HPMC is preferable. TC-5 (Japanese Pharmacoepia hydroxypropyl methylcellulose 2910), for example, is used as HPMC.

The formulation may further contain an ingredient commonly used in the pharmaceutical field. Examples of the ingredient include, but not limited to, a diluent, a disintegrant, a binder, a lubricant, a colorant, a pH adjusting agent, a surfactant, a stabilizing agent, a sour agent, a flavor, a glidant and the like. These ingredients are used in an amount commonly used in the pharmaceutical field unless otherwise specifically stated.

Examples of the diluent include, but not limited to, a sugar or a sugar alcohol such as lactose (for example, lactose monohydrate), fructose, glucose, mannitol or sorbitol; a starch such as corn starch, potato starch, wheat starch, rice starch, partially pregelatinized starch, pregelatinized starch or porous starch; microcrystalline cellulose; anhydrous calcium phosphate, precipitated calcium carbonate, calcium silicate and the like.

In the formulation, the diluent is preferably used in an amount to give a content of from 5 to 95% by weight based on 100 parts by weight of the formulation of the invention.

Examples of a disintegrant include, but not limited to, carboxymethyl cellulose, carboxymethyl cellulose calcium, carboxymethyl starch sodium, croscarmellose sodium, carmellose calcium, crospovidone, low-substituted hydroxypropyl cellulose, and hydroxypropyl starch. The amount of the disintegrant can be an amount to give a content of from 0.5 to 25 parts by weight based on 100 parts by weight of the scored tablet of the invention.

Examples of a binder include, but not limited to, hydroxypropyl cellulose (for example, grade: L, SL, SSL (trade name); Nippon Soda Co., Ltd.), hydroxypropylmethyl cellulose (for example, hypromellose 2910 (for example, TC-5 (grade: MW, E, EW, R, RW) (trade name); Shin-Etsu Chemical Co., Ltd.)), polyvinylpyrrolidone (povidone), and gum arabic.

In the formulation, the binder is preferably used in an amount to give a content of from 1 to 20% by weight based on 100 parts by weight of the scored tablet of the invention.

Preferred examples of the lubricant include, but not limited to, magnesium stearate, calcium stearate, talc, sucrose fatty acid ester, sodium stearyl fumarate and the like. The used amount of the lubricant is an amount to give a content of from 0.5 to 2% by weight based on 100 parts by weight of the solid preparation.

Preferred examples of the colorant include, but not limited to, a food dye (for example, food yellow No. 5, food red No. 2, food blue No. 2), a food lake color, iron oxide red, and iron oxide yellow.

Preferred examples of the pH adjusting agent include, but not limited to, citrate, phosphate, carbonate, tartrate, fumarate, acetate and amino acid salt.

Preferred examples of the surfactant include, but not limited to, sodium lauryl sulfate, polysorbate 80, and polyoxyethylene (160) polyoxypropylene (30) glycol.

Preferred examples of the stabilizing agent include, but not limited to, tocopherol, tetrasodium edetate, nicotinamide, and a cyclodextrin.

Preferred examples of the sour agent include, but not limited to, ascorbic acid, citric acid, tartaric acid and malic acid.

Preferred examples of the flavor include, but not limited to, menthol, Mentha oil, lemon oil and vanillin.

Preferred examples of the glidant include, but not limited to, colloidal silicon dioxide, aqueous silicon dioxide and talc.

The above ingredients may be used by combining two or more members at an appropriate ratio.

The formulations disclosed herein include a composition for daily administration. In some embodiments, the therapeutic effect is maintained with one unit, twice daily. In some embodiments, the therapeutic effect is maintained with one unit, three times daily. In some such embodiments, the duration of each dose's effect is between four (4) to six (6) hours.

The suppository formulation of the invention can be of any suitable form, for example, a gel, a capsule, or a tablet. The suppository formulation of the invention can be of any suitable shape, for example, round, conical, and torpedo.

In another aspect, the present invention is directed to a method for treating a disease in a subject, the method comprising administering a formulation as described herein. In certain embodiments, the method comprises rectally administering the formulation. In other embodiments, the method comprises vaginally administering the formulation. Examples of a disease or a disorder that can be treated by the invention include, but are not limited to, a rectal disease, a vaginal disease, pain associated with cancer, neuropathic pain and HIV-associated sensory neuropathy, side effects of chemotherapy including nausea and pain, symptoms of neurology and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis and dystonia.

Examples of a rectal disease include, but not limited to, hematochezia, hemorrhoid, anal fissure, rectocele, ulcerative colitis, rectal infection (e.g., infection by Lymphogranuloma venereum), rectal cancer, fetal incontinence, and pruritus ani.

Examples of a vaginal disease include, but not limited to, herpes genitalis, gonorrhea, chlamydia, trichomoniasis, genital wart, human papillomavirus (HPV) disease, candidal vulvovaginitis, bacterial vaginosis (BV), vaginismus, vaginal obstruction, vaginal hypoplasia, vaginal cancer, cervical cancer, persistent genital arousal disorder, vaginal prolapse, vulvodynia, vaginal discharge, sores, and vaginitis. In certain embodiments, the vaginal disease is herpes genitalis, gonorrhea, chlamydia, trichomoniasis, genital wart, human papillomavirus (HPV) disease, candidal vulvovaginitis, bacterial vaginosis (BV), vaginismus, vaginal obstruction, vaginal hypoplasia, vaginal cancer, cervical cancer, persistent genital arousal disorder, vaginal prolapse, vulvodynia, vaginal discharge, soreness, and vaginitis.

In some embodiments where the disorder is cancer, pain associated with cancer; nausea associated with chemotherapy; or a combination thereof, the composition described herein exerts reduced hallucinatory effects compared to smoking a cannabis containing cigarette or ingesting a cannabis containing foodstuff with the same amount of active ingredients.

In yet another aspect, provided herein is a method for the manufacture of a suppository formulation. The method may include the steps of: providing one or more cannabinoids; providing one or more moldable polymers; and blending or mixing said one or more cannabinoids with said one or more moldable polymers. These steps are carried out based on the techniques and processes known to one of skilled in the art. In certain embodiments, the method comprises the steps of: providing one or more cannabinoids; providing one or more moldable polymers; and mixing said one or more cannabinoids with said one or more moldable polymers. In other embodiments, the method comprises mixing one or more cannabinoids with one or more moldable polymers.

All patents and literature references cited in the present specification are hereby incorporated by reference in their entirety.

The present invention will be specifically explained by way of examples, but these examples are not intended to limit the present invention.

### EXAMPLE 1

### Suppository Formulation Having Cannabinoid

Exemplary formulations described above are shown in Tables 1-4 and are prepared using the methods described herein.

The formulation is a therapeutic suppository for rectal or vaginal administration. The suppository includes a mixture of a therapeutic agent (e.g., cannabinoid extract) and a moldable polymer. The moldable polymer includes a mixture of polyethylene glycols of various molecular weights. Specifically, the moldable polymer includes a mixture of PEG 1450 and PEG 4000.

The formulation is suitable for rectal or vaginal insertion.

TABLE 1
The exemplary suppository formulation
having CBD and THC cannabinoid extract

| | Concentration |
|---|---|
| Ingredients | (% w/w) |
| CBD and THC cannabinoid extract | 2.0 |
| PEG 1450 | 73.5 |
| PEG 4000 | 24.5 |
| Total | 100 |

TABLE 2
The exemplary suppository formulation having
CBDa and THCa cannabinoid extract

| | Concentration |
|---|---|
| Ingredients | (% w/w) |
| CBDa and THCa cannabinoid extract | 2.0 |
| PEG 1450 | 73.5 |
| PEG 4000 | 24.5 |
| Total | 100 |

TABLE 3
The exemplary suppository formulation
having CBD or CBDa cannabinoid extract

| | Concentration |
|---|---|
| Ingredients | (% w/w) |
| CBD or CBDa cannabinoid extract | 2.0 |
| PEG 1450 | 73.5 |
| PEG 4000 | 24.5 |
| Total | 100 |

TABLE 4
The exemplary suppository formulation
having THC or THCa cannabinoid extract

| | Concentration |
|---|---|
| Ingredients | (% w/w) |
| THC or THCa cannabinoid extract | 2.0 |
| PEG 1450 | 73.5 |
| PEG 4000 | 24.5 |
| Total | 100 |

Unless defined otherwise, all technical and scientific terms used herein have the same meanings as commonly understood by one of ordinary skill in the art.

Having described preferred embodiments of the present invention with reference to the accompanying drawings, it is to be understood that the present invention is not limited to the above-mentioned embodiments and that various changes and modifications can be effected by one skilled in the art without departing from the spirit or scope of the present invention as defined in the appended claims.

What is claimed is:

1. A suppository formulation comprising: at least one cannabinoid or a derivative thereof and at least one moldable polymer.

2. The formulation of claim 1, wherein said suppository formulation is an insertable or implantable formulation in a subject.

3. The formulation of claim 1, wherein said suppository formulation is a rectal suppository formulation.

4. The formulation of claim 1, wherein said suppository formulation is a vaginal suppository formulation.

5. The formulation of claim 1, wherein said cannabinoid is an extract from a cannabis plant.

6. The formulation of claim 1, wherein the formulation has a combination of at least two cannabinoids.

7. The formulation of claim 6, wherein the two cannabinoids are selected from Tetrahydrocannabinolic acid (THCa), Cannabidiolic acid (CBDa), Cannabinolic acid (CBNa), Cannabichromenic acid (CBCa), Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM) and derivatives thereof.

8. The formulation of claim 6, wherein at least two cannabinoids are in a 1:1 proportion by weight.

9. The formulation of claim 6, wherein at least two cannabinoids are in a 10:1 proportion by weight.

10. The formulation of claim 6, wherein at least two cannabinoids are in a 20:1 proportion by weight.

11. The formulation of claim 6, wherein two cannabinoids are THC and CBD.

12. The formulation of claim 6, wherein two cannabinoids are THCa and CBDa.

13. The formulation of claim 1, wherein at least one cannabinoid is THC or THCa present in an amount ranging from about 0.1 mg to about 200 mg.

14. The formulation of claim 1, wherein at least one cannabinoid is CBD or CBDa present in an amount ranging from about 0.01 mg to about 200 mg.

15. The formulation of claim 1, wherein said polymer is a polyethylene glycol (PEG).

16. The formulation of claim 15, wherein said PEG is characterized by a molecular weight that ranges from about 1000 to 5000.

17. The formulation of claim 15, wherein said PEG is PEG 1450.

18. The formulation of claim 15, wherein said PEG is PEG 4000.

19. The formulation of claim 1, wherein the formulation has a combination of at least two polymers.

20. The formulation of claim 19, wherein the first polymer is PEG 1450 and the second polymer is PEG 4000.

21. The formulation of claim 20, wherein said PEG 1450 and said PEG 4000 are present at a ratio ranging from about 2:1 to about 5:1.

22. The formulation of claim 20, wherein said PEG 1450 and said PEG 4000 are present at a ratio of 3:1.

23. The formulation of claim 1, wherein the formulation further comprises a terpene.

24. The formulation of claim 23, wherein said terpene is selected from the group consisting of beta-myrcene, limonene, beta caryopyllene, caryopyllene oxide, terpineol, citronellol, linalool, humulene, beta-amyrin, and cycloartenol.

25. The formulation of claim 1, wherein the formulation further comprises a pharmaceutically accepted carrier.

26. The formulation of claim 25, wherein the pharmaceutically accepted carrier is selected from a group consisting of cellulose, microcrystalline cellulose, silicified microcrystalline cellulose, starch, pregelatinized starch, dicalcium phosphate, tricalcium phosphate, and mixtures thereof.

27. The formulation of claim 25, wherein the pharmaceutically accepted carrier is microcrystalline cellulose.

28. The formulation of claim 1, wherein the formulation further comprises a solvent.

29. The formulation of claim 28, wherein the solvent is selected from a group consisting of ethanol, methanol, isopropanol, chloroform, propylene glycol, polyethylene glycol, glycerine, limonene, myrcene, linalool, alpha bisabolol, delta 3 carene, borneol, alpha-pinene, beta-pinene, eucalyptol, terpineol, caryophyllene, camphene, or combinations thereof.

30. The formulation of claim 28, wherein the solvent is ethanol.

31. A method for treating a disease in a subject, the method comprising administering a formulation according to any one of claims 1-30.

32. The method of claim 31, wherein the disease is selected from a group consisting of a rectal disease, a vaginal disease, pain associated with cancer, neuropathic pain and HIV-associated sensory neuropathy, side effects of chemotherapy including nausea and pain, symptoms of neurology and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis and dystonia.

33. The method of claim 31, wherein said administration is a rectal administration.

34. The method of claim 31, wherein said administration is a vaginal administration.

35. The method of claim 32, wherein said rectal disease is hematochezia, hemorrhoid, anal fissure, rectocele, ulcerative colitis, rectal infection by Lymphogranuloma venereum, rectal cancer, fetal incontinence, or pruritus ani.

36. The method of claim 32, wherein said vaginal disease is herpes genitalis, gonorrhea, chlamydia, trichomoniasis, genital wart, human papillomavirus (HPV) disease, candidal vulvovaginitis, bacterial vaginosis (BV), vaginismus, vaginal obstruction, vaginal hypoplasia, vaginal cancer, cervical cancer, persistent genital arousal disorder, vaginal prolapse, vulvodynia, vaginal discharge, soreness, or vaginitis.

37. A method for the manufacture of a suppository formulation of claim 1, the method comprising the steps of:
providing one or more cannabinoids;
providing one or more moldable polymers; and
mixing said one or more cannabinoids with said one or more moldable polymers.
